(19) United States
(12) Patent Application Publication
(10) Pub. No.: US 2026/0273250 A1
(43) Pub. Date: Sep. 17, 2026

(54) DRUG DELIVERY ASSEMBLY FOR EXTENDED DRUG DELIVERY AND TUNABILITY

(71) Applicant: Mott Corporation, Farmington, CT (US)

(72) Inventors: Alex Hill, Manchester, CT (US); Aravind Mohanram, Avon, CT (US); David Gonthier, Middletown, CT (US)

(21) Appl. No.: 19/673,086

(22) Filed: May 11, 2026

Related U.S. Application Data
(62) Division of application No. 17/886,585, filed on Aug. 12, 2022.
(60) Provisional application No. 63/233,013, filed on Aug. 13, 2021.

(51) Int. Cl. A61M 31/00; A61K 9/00; A61M 5/142; A61M 39/02

(52) U.S. Cl. CPC A61M 31/002; A61K 9/0024; A61M 5/14276; A61M 2039/027

(57) ABSTRACT

Disclosed herein are advantageous drug delivery assemblies, and related methods of fabrication and use thereof. The present disclosure provides improved drug delivery assemblies for extended drug delivery (e.g., via passive diffusion) and/or tunability, and improved systems/methods for utilizing and fabricating the drug delivery assemblies. More particularly, the present disclosure provides single or dual compartment, and dual porous membrane based (e.g., porous zinc membrane based) drug delivery assemblies for extended drug delivery (e.g., via passive diffusion) and/or tunability. The present disclosure also provides for a method for utilizing a drug delivery assembly. The assemblies can be utilized for the extended drug delivery of pharmaceuticals or the like, such as biologics, for the sustained release of medication for greater than six months to overcome difficulties with daily dosing regimes.

### CROSS-REFERENCE TO RELATED APPLICATIONS

This application is a Divisional of U.S. application Ser. No. 17/886,585, filed on Aug. 12, 2022, which claims priority to U.S. Provisional Application No. 63/233,013, which was filed on Aug. 13, 2021, the entire contents of each of which is incorporated herein by reference.

### FIELD OF THE DISCLOSURE

The present disclosure relates to drug delivery assemblies for extended drug delivery and/or tunability and systems/methods for utilizing and fabricating the drug delivery assemblies and, more particularly, to single or dual compartment, and dual porous membrane based (e.g., porous zinc membrane based) drug delivery assemblies for extended drug delivery (e.g., via passive diffusion) and/or tunability.

### BACKGROUND OF THE DISCLOSURE

In general, some drug delivery assemblies or the like are known.

An interest exists for improved drug delivery assemblies, and related methods of use.

These and other inefficiencies and opportunities for improvement are addressed and/or overcome by the assemblies, methods and devices of the present disclosure.

### BRIEF SUMMARY OF THE DISCLOSURE

The present disclosure provides advantageous drug delivery assemblies for extended drug delivery and/or tunability, and improved systems/methods for utilizing and fabricating the drug delivery assemblies. More particularly, the present disclosure provides single or dual compartment, and dual porous membrane based (e.g., porous zinc membrane based) drug delivery assemblies for extended drug delivery (e.g., via passive diffusion) and/or tunability.

The present disclosure provides for a drug delivery assembly including a housing that extends from a first end to a second end, the housing defining a first compartment and a second compartment; a first opening in the housing in communication with the first and second compartments, the first opening located at a position between the first and second ends of the housing; a second opening in the housing, the second opening positioned at the second end of the housing, with the second opening in communication with an area that is external to the housing; and a first porous membrane positioned in the first opening, and a second porous membrane positioned in the second opening.

The present disclosure also provides for a drug delivery assembly wherein the first and second porous membranes are fabricated out of zinc, magnesium or iron or combinations thereof.

The present disclosure also provides for a drug delivery assembly wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biomaterials or combinations thereof.

The present disclosure also provides for a drug delivery assembly wherein the housing is substantially tubular or substantially cylindrical.

The present disclosure also provides for a drug delivery assembly wherein the housing is fabricated from a metal or from plastic.

The present disclosure also provides for a drug delivery assembly wherein the housing is fabricated from a material selected from the group consisting of zinc, iron, magnesium, titanium, metal alloys, polylactic acid, poly(lactic-co-glycolic acid) or combinations thereof.

The present disclosure also provides for a drug delivery assembly wherein the area that is external to the housing includes subcutaneous tissue.

The present disclosure also provides for a drug delivery assembly wherein the first porous membrane has a smaller pore size relative to a pore size of the second porous membrane.

The present disclosure also provides for a drug delivery assembly wherein a range of a mean pore size of the first porous membrane is between 0.05 to 20 μm; and a range of a mean pore size of the second porous membrane is between 1 to 100 μm.

The present disclosure also provides for a drug delivery assembly wherein the first and second porous membranes are fabricated out of titanium or polyether ether ketone.

The present disclosure also provides for a drug delivery assembly wherein the first and second porous membranes are in the shape of a flat cylinder or a thin needle.

The present disclosure also provides for a drug delivery assembly wherein the first and second compartments are configured and dimensioned to house drug or active agent particles.

The present disclosure also provides for a drug delivery assembly wherein an internal volume of the first compartment is about 0.10 to 2.5 mL, and wherein an internal volume of the second compartment is about 0.10 to 2.5 mL.

The present disclosure also provides for a drug delivery assembly wherein a total internal volume of the first compartment and of the second compartment added together ranges from about 0.20 mL to about 5 mL.

The present disclosure also provides for a drug delivery assembly wherein the diameter of the housing is about 0.20 to 15 mm, and the length of the housing is about 0.50 to 15 cm.

The present disclosure also provides for a drug delivery assembly including a housing that extends from a first end to a second end, the housing defining a first compartment; an opening in the housing in communication with an area that is external to the housing; a first porous membrane positioned proximal to the opening; and a second porous membrane positioned in the opening.

The present disclosure also provides for a drug delivery assembly wherein the first and second porous membranes are joined or attached together.

The present disclosure also provides for a drug delivery assembly wherein the first and second porous membranes are fabricated out of biodegradable metals, such as zinc, magnesium or iron or combinations thereof.

The present disclosure also provides for a drug delivery assembly wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biocompatible materials, biodegradable materials, or combinations thereof.

The present disclosure also provides for a drug delivery assembly wherein the first porous membrane has a smaller pore size relative to a pore size of the second porous membrane.

The present disclosure also provides for a drug delivery assembly wherein a range of a mean pore size of the first porous membrane is between 0.05 to 20 μm; and a range of a mean pore size of the second porous membrane is between 1 to 100 μm.

The present disclosure also provides for a drug delivery assembly wherein the first compartment is configured and dimensioned to house drug or active agent particles.

The present disclosure also provides for a method for utilizing a drug delivery assembly including providing a housing that extends from a first end to a second end, the housing defining a first compartment and a second compartment; providing a first opening in the housing in communication with the first and second compartments, the first opening located at a position between the first and second ends of the housing; providing a second opening in the housing, the second opening positioned at the second end of the housing, with the second opening in communication with an area that is external to the housing; and positioning a first porous membrane in the first opening, and a positioning a second porous membrane in the second opening; and providing drug or active agent particles to the first or second compartment.

The present disclosure also provides for a method for utilizing a drug delivery assembly wherein the first and second porous membranes are fabricated out of zinc, magnesium or iron.

The present disclosure also provides for a method for utilizing a drug delivery assembly wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biomaterials or combinations thereof.

The present disclosure also provides for a drug delivery assembly including a housing that extends from a first end to a second end, the housing defining a first compartment; an opening in the housing in communication with an area that is external to the housing; a first porous membrane positioned proximal to the opening and secured to the housing via a first end cap; and a textured section on the housing, the textured section including raised textures on the housing of from 200 to 500 microns.

The present disclosure also provides for a drug delivery assembly wherein the first end cap includes a hood-like feature designed to prevent fibrosis from impeding the diffusion properties of the first porous membrane.

The present disclosure also provides for a drug delivery assembly further including a second porous membrane secured to the first end of the housing via a second end cap.

The present disclosure also provides for a drug delivery assembly wherein the first compartment is configured and dimensioned to house drug or active agent particles; and wherein the area that is external to the housing includes subcutaneous tissue; and wherein a range of a mean pore size of the first porous membrane is between 0.10 to 100 μm.

The present disclosure also provides for a drug delivery assembly wherein the housing is fabricated from zinc or titanium.

The present disclosure also provides for a drug delivery assembly further including a septum member secured to the first end of the housing via a second end cap.

The present disclosure also provides for a drug delivery assembly further including a drug loading port positioned at the first end of the housing.

The present disclosure also provides for a drug delivery assembly wherein the first end of the housing is closed off.

The present disclosure also provides for a drug delivery assembly wherein the textured section on the housing comprises a grooved or threaded section, the grooved or threaded section having a plurality of tissue grooves.

The above described and other features are exemplified by the following figures and detailed description.

Any combination or permutation of embodiments is envisioned. Additional advantageous features, functions and applications of the disclosed assemblies, methods and devices of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are exemplary embodiments wherein the like elements are numbered alike.

Features and aspects of embodiments are described below with reference to the accompanying drawings, in which elements are not necessarily depicted to scale.

Exemplary embodiments of the present disclosure are further described with reference to the appended figures. It is to be noted that the various features, steps, and combinations of features/steps described below and illustrated in the figures can be arranged and organized differently to result in embodiments which are still within the scope of the present disclosure. To assist those of ordinary skill in the art in making and using the disclosed assemblies, methods and devices, reference is made to the appended figures, wherein:

FIG. 1 is a side cross-sectional view of an exemplary drug delivery assembly, according to the present disclosure.

FIG. 2 is a modeled delivery profile of the drug delivery assembly of FIG. 1 and for an exemplary drug.

FIG. 3 is a modeled release profile of drug showing percent released over time for an exemplary drug delivery assembly according to the present disclosure.

FIG. 4 is a side cross-sectional view of another exemplary drug delivery assembly, according to the present disclosure.

FIG. 5 is a side view of another exemplary drug delivery assembly, according to the present disclosure.

FIG. 6 is an exploded partial view of the drug delivery assembly of FIG. 5.

FIG. 7 is an exploded side view of the drug delivery assembly of FIG. 5.

FIG. 8 is a side perspective view of the drug delivery assembly of FIG. 5.

FIG. 9 is a cross-sectional side view of another exemplary drug delivery assembly, according to the present disclosure.

FIG. 10 is a side perspective view of another exemplary drug delivery assembly, according to the present disclosure.

FIG. 11 is an exploded side view of the drug delivery assembly of FIG. 10, and showing the bottom port area in a side cross-sectional view.

FIG. 12 is a cross-sectional side view of another exemplary drug delivery assembly, according to the present disclosure.

### DETAILED DESCRIPTION OF THE DISCLOSURE

The exemplary embodiments disclosed herein are illustrative of advantageous drug delivery assemblies, and systems of the present disclosure and methods/techniques thereof. It should be understood, however, that the disclosed embodiments are merely exemplary of the present disclosure, which may be embodied in various forms. Therefore, details disclosed herein with reference to exemplary drug delivery assemblies and associated processes/techniques of assembly and use are not to be interpreted as limiting, but merely as the basis for teaching one skilled in the art how to make and use the advantageous drug delivery assemblies and/or alternative drug delivery assemblies of the present disclosure.

Disclosed herein are advantageous drug delivery assemblies, and related methods of fabrication and use thereof.

The present disclosure provides improved drug delivery assemblies for extended drug delivery (e.g., via passive diffusion) and/or tunability, and improved systems/methods for utilizing and fabricating the drug delivery assemblies.

More particularly, the present disclosure provides single or dual compartment, and dual porous membrane based (e.g., porous zinc membrane based) drug delivery assemblies for extended drug delivery (e.g., via passive diffusion) and/or tunability.

Referring now to the drawings, like parts are marked throughout the specification and drawings with the same reference numerals, respectively. Drawing figures are not necessarily to scale and in certain views, parts may have been exaggerated for purposes of clarity.

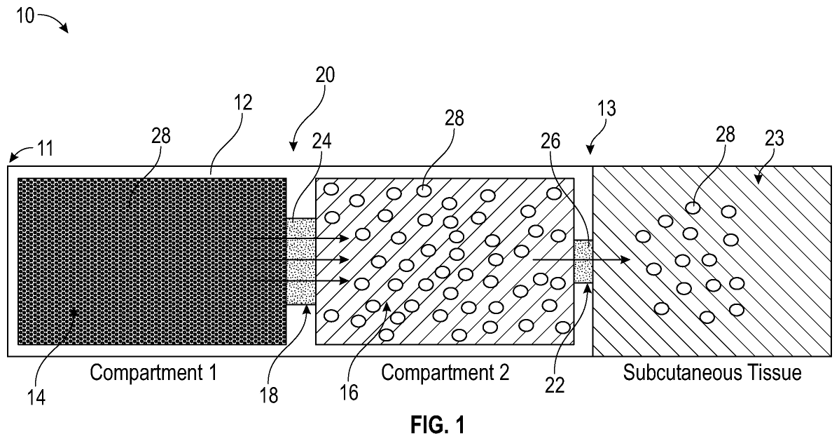

As shown in FIG. 1, there is illustrated a drug delivery assembly 10 depicting an embodiment of the present disclosure.

Exemplary drug delivery assembly 10 takes the form of a dual compartment and dual porous membrane based (e.g., porous zinc membrane based) drug delivery assembly 10 for extended drug 28 delivery (e.g., via passive diffusion) and/or tunability or the like, although the present disclosure is not limited thereto.

As shown in FIG. 1, drug delivery assembly 10 includes a housing 12 that extends from a first end 11 to a second end 13. In exemplary embodiments, the housing 12 is substantially tubular or substantially cylindrical, although the present disclosure is not limited thereto. Rather, it is noted that housing 12 can take a variety of shapes and/or forms.

It is noted that housing 12 can be fabricated from a variety of materials. For example, housing 12 can be fabricated from a metal (e.g., magnesium, zinc, titanium, iron, stainless steel, or an alloy thereof). Housing 12 can also be fabricated from a biodegradable plastic (e.g., PLA or PLLA, etc.). It is noted that the housing can be fabricated from a material selected from the group consisting of zinc, iron, magnesium, titanium, metal alloys, polylactic acid, poly(lactic-co-glycolic acid), or combinations thereof.

In non-limiting examples, housing 12 takes the form of a tube shape, with an overall length of 0.5 to 25 cm, preferably 0.5 to 10 cm, more preferably between 1 to 5 cm. Housing 12 can have a diameter between 1 to 25 mm, preferably between 2 to 5 mm. The housing 12 can have a wall thickness between 1 to 5 mm. It is noted that the diameter of the housing can be about 3 to 7 mm, and the length of the housing can be about 3 to 12.5 mm.

Exemplary housing 12 defines a first compartment 14 and a second compartment 16, with a first opening 18 in housing 12 in communication with the first and second compartments 14, 16. The first opening 18 is located at a position 20 (e.g., an intermediate position 20) between the first and second ends 11, 13 of housing 12.

A second opening 22 in housing 12 is positioned at the second end 13 of housing 12 of assembly 10, as shown in FIG. 1. In general, second opening 22 can be in communication with an area 23 that is external to the housing 12 of assembly 10 (e.g., an area 23 such as, for example, subcutaneous tissue of a body of a patient, after assembly 10 is positioned in the patient).

In exemplary embodiments, a first porous membrane 24 is positioned in the first opening 18, and a second porous membrane 26 is positioned in the second opening 22.

It is noted that the first porous membrane 24 can be attached and/or bonded relative to the first opening 18 of housing 12 and that the second porous membrane 26 can be attached and/or bonded relative to the second opening 18 of housing 12 via various attachment or bonding methods (e.g., sintering bonding, adhesive, press-fit, etc.).

FIG. 1 shows a cross section of housing 12 of assembly 10 (e.g., tubular assembly 10), with the assembly 10 having first compartment 14 in fluid communication with second compartment 16 via first porous membrane 24 positioned in first opening 18, and with the assembly 10 having second compartment 16 in fluid communication with area 23 via second porous membrane 26 positioned in second opening 22. Each interior cavity of first and/or second compartments 14, 16 can be filled with a drug solution 28 or the like (e.g., based on a formulation a user).

In exemplary embodiments, the first porous membrane 24 is generally finer, having a smaller pore size relative to second porous membrane 26, although the present disclosure is not limited thereto.

An exemplary range of the mean pore size of the first porous membrane 24 can be between 0.05 to 20 μm, preferably between 0.2 to 1 μm. However, it is noted that the mean pore size of the first porous membrane 24 can be as large as 20 μm, or as large as 100 μm, or possibly even larger.

The second porous membrane 26 is generally coarser relative to the first porous membrane 24, with the second porous membrane 26 generally having a larger pore size than the first porous membrane 24, although the present disclosure is not limited thereto.

An exemplary range of the mean pore size of the second porous membrane 26 can be between 1 to 100 μm, although the present disclosure is not limited thereto. In some embodiments, it is noted that mean pore size of the first porous membrane 24 can be substantially the same as or similar to the mean pore size of the second porous membrane 26.

In exemplary embodiments, the coarseness and microstructure of the second porous membrane 26 can prevent bio-fouling of assembly 10, whereas the tightness of the first porous membrane 24 can allow for precision dosage/control over diffusion/drug 28 delivery to eventual area 23.

In non-limiting examples, each porous membrane 24, 26 utilized to regulate the mass transfer of the drug 28 can be fabricated from zinc, magnesium, iron, titanium, polyether ether ketone, or another applicable or suitable biomaterial. Each porous membrane 24, 26 can be in the shape of a flat cylinder or thin needle, with diameters between 0.25 to 10 mm and thicknesses between 0.25 to 10 mm, although the present disclosure is not limited thereto.

In general, each compartment 14, 16 is configured and dimensioned to house drug or active agent particles 28.

As such, FIG. 1 depicts an exemplary drug delivery assembly 10, with the assembly 10 having first and second compartments 14, 16, and having first and second porous membranes 24, 26 to regulate the delivery of a compound/drug or active agent particles 28 to area 23 that is external to assembly 10.

Exemplary assembly 10 includes first and second compartments 14, 16 separated by the first porous membrane 24. It is noted that the second compartment 16 has two openings, that is, first opening 18 joining the first and second compartments 14, 16, and second opening 22 that is in communication with area 23.

In exemplary embodiments, it is noted that the assembly 10 is configured and dimensioned to be implanted in a body of a patient or the like (e.g., the subcutaneous layer of a body of a patient or the like). It is also noted that the assembly 10 can be located in other/different areas of the body or the like.

Exemplary assembly 10 is an improvement over other conventional designs/assemblies, as the dual compartment 14, 16 feature of assembly 10 has been shown via modeling to be able to deliver the medication 28 within therapeutic concentrations over longer periods of time to area 23, and to be able to deliver a larger percentage of the loaded dose 28 within those same concentrations.

It is noted that some conventional approaches for extended drug release include an osmotic pump and some polymeric based solutions that deliver medication for an extended period of time up to thirty to 120 days (polymer matrix systems), and 180 to 360 days (osmotic pump systems). The passive diffusion based drug 28 delivery of exemplary assembly 10 is able to deliver medication for 180 days or longer. Moreover, this exemplary assembly 10 can increase the percent of the initial dose delivered while extending the time spent in a therapeutic concentration window while still maintaining a small footprint.

Furthermore, the porous membrane 24 separating compartment 16 from the external area 23 can be much coarser than the other porous membrane 24 of assembly 10. This can allow for the retention of precise drug delivery with the finer media of membrane 24, and can prevent bio-fouling as the coarser porous membrane 26 is inherently resistant, as determined by testing, to the formation of protein films (e.g., on second end 13).

In an example embodiment, the internal volume of housing 12 can be about 1 mL, with the internal volume divided into a 0.6 mL compartment in first compartment 14, and with a 0.4 mL compartment in second compartment 16. It is noted that a total internal volume of the first compartment and of the second compartment (added together) can range from about 0.10 mL to 5 mL, preferably between 0.10 to 2 mL.

The overall diameter of housing 12 of assembly 10 can be about 7 mm, and the length of housing 12 of assembly 10 can be about 4 cm.

In an example embodiment, the first porous membrane 24 can be classified as a Media Grade (“MG”) 0.1 having a mean pore size of 0.1 μm. The diameter of this exemplary first porous membrane 24 can be about 0.5 mm by 1 mm thick. The second porous membrane 26 can be classified as a MG 2, with a diameter of 1.5 mm by 3 mm thick. The membranes 24, 26 can be fabricated out of 99.99% pure zinc, although the present disclosure is not limited thereto.

In use, exemplary assembly 10 can be utilized for the extended drug 28 delivery of pharmaceuticals or the like, such as biologics, proteins and small molecules (100 to 1,000 g/mol) for the sustained release of medication for greater than six months to overcome difficulties with daily dosing regimes.

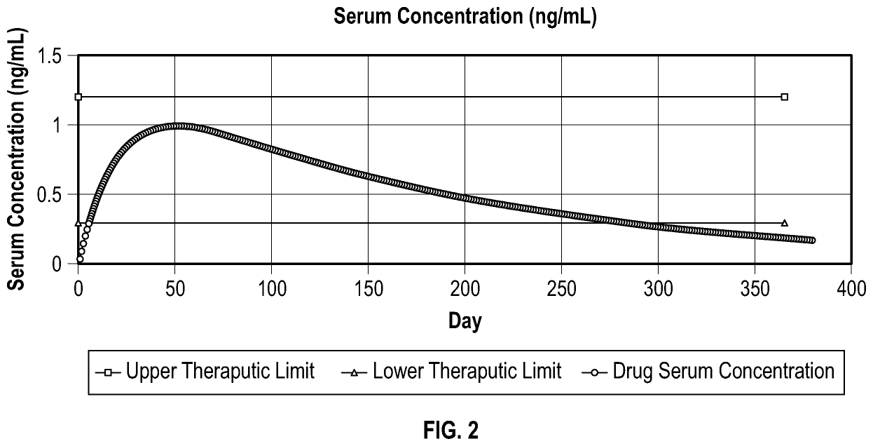

In FIG. 2, the release profile of a drug 28 through an exemplary assembly 10 is shown. For this example, the drug 28 was only loaded in the first compartment 14 (behind the first membrane 24), but the drug 28 could be loaded in both compartments 14, 16 theoretically. In FIG. 2, the y-axis details serum concentration, or a description of how concentrated the drug 28 is predicted to be in the blood after x days. The orange and gray lines detail a concentration window where the serum concentration indicates that the drug is effective. The exemplary assembly 10 having compartments 14, 16 allows for an extension of the time spent in this window by better regulating the diffusion gradient between the second compartment 16 and the area 23 by introducing an intermediate volume (compartment 16) and membrane 24 to facilitate mass transfer.

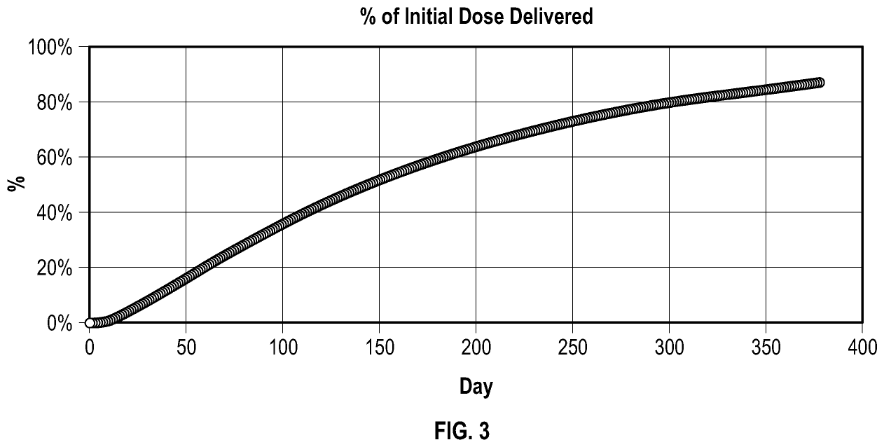

In FIG. 3, the predicted percent released over time relationship for an assembly 10 having compartments 14, 16 is shown. A goal of a drug delivery assembly is to have the percent released close to 90% or greater at the time which the serum concentration is modeled to be below the minimum effective therapeutic level. An assembly 10 having compartments 14, 16, via a better control over the drug delivery physics described above, allows for the achievement of a higher percent delivered at this time.

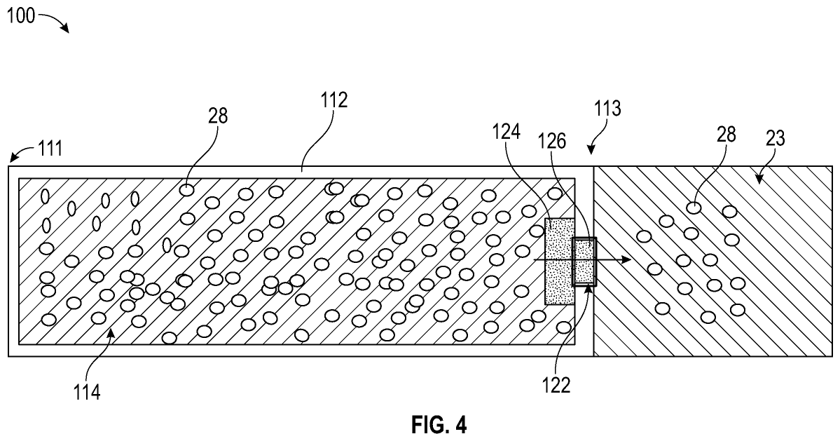

In another embodiment and as shown in FIG. 4, exemplary drug delivery assembly 100 takes the form of a single compartment and dual porous membrane based (e.g., porous zinc membrane based) drug delivery assembly 100 for extended drug 28 delivery (e.g., via passive diffusion) and/or tunability or the like.

Exemplary drug delivery assembly 100 includes a housing 112 that extends from a first end 111 to a second end 113. In exemplary embodiments, the housing 112 is substantially tubular or substantially cylindrical, although the present disclosure is not limited thereto. Rather, it is noted that housing 112 can take a variety of shapes and/or forms.

It is noted that housing 112 can be fabricated from a variety of materials. For example, housing 112 can be fabricated from a metal (e.g., magnesium, zinc, iron, stainless steel, or an alloy thereof). Housing 112 can also be fabricated from a biodegradable plastic (e.g., PLA or PLLA, etc.).

In non-limiting examples, housing 112 takes the form of a tube shape, as similarly discussed above relative to housing 12 of assembly 10.

Exemplary housing 112 defines a first compartment 114. An opening 122 in housing 112 is positioned at the second end 113 of housing 112 of assembly 100, as shown in FIG. 4. In general, opening 122 can be in communication with an area 23 that is external to the housing 112 of assembly 100 (e.g., an area 23 such as, for example, subcutaneous tissue of a body of a patient, after assembly 100 is positioned in the patient).

In exemplary embodiments, a first porous membrane 124 is positioned proximal to the opening 122, and a second porous membrane 126 is positioned substantially within the opening 122, as discussed further below.

It is noted that the first porous membrane 124 can be attached and/or bonded relative to the opening 122 of housing 112 (and/or to membrane 126) and that the second porous membrane 126 can be attached and/or bonded relative to the opening 122 of housing 112 (and/or to membrane 124) via various attachment or bonding methods (e.g., sintering bonding, adhesive, press-fit, etc.).

Interior cavity of first compartment 114 can be filled with a drug solution 28 or the like (e.g., based on a formulation a user).

In exemplary embodiments, the first porous membrane 124 is generally finer, having a smaller pore size relative to second porous membrane 126. In certain embodiments, the first porous membrane 124 has a smaller mean pore size than the second porous membrane 126.

An exemplary range of the mean pore size of the first porous membrane 124 can be between 0.05 to 1.0 μm. The second porous membrane 126 is generally coarser relative to the first porous membrane 124, with the second porous membrane 126 generally having a larger pore size than the first porous membrane 124.

An exemplary range of the mean pore size of the second porous membrane 126 can be between 1 to 100 μm, although the present disclosure is not limited thereto.

In exemplary embodiments, these two membranes 124, 126, are joined or attached together to form one continuous body, leaving a porous matrix 124 and 126 with a gradient pore size structure.

In exemplary embodiments, the coarseness of the second porous membrane 126 can prevent bio-fouling of assembly 100, whereas the tightness of the first porous membrane 124 can allow for precision dosage/control over diffusion/drug 28 delivery to eventual area 23.

In non-limiting examples, each porous membrane 124, 126 utilized to regulate the mass transfer of the drug 28 can be fabricated from zinc, titanium, polyether ether ketone, or another applicable or suitable biomaterial. Each porous membrane 124, 126 can be in the shape of a flat cylinder or thin needle, with diameters between 0.25 to 5 mm and thicknesses between 0.25 to 10 mm, although the present disclosure is not limited thereto.

In general, compartment 114 is configured and dimensioned to house drug or active agent particles 28.

As such, FIG. 4 depicts an exemplary drug delivery assembly 100, with the assembly 100 having compartment 114, and having first and second porous membranes 124, 126 to regulate the delivery of a compound/drug or active agent particles 28 to area 23 that is external to assembly 100.

In exemplary embodiments, it is noted that the assembly 100 is configured and dimensioned to be implanted in a body of a patient or the like (e.g., the subcutaneous layer of a body of a patient or the like). It is also noted that the assembly 100 can be located in other/different areas of the body or the like.

In use, exemplary assembly 100 can be utilized for the extended drug 28 delivery of pharmaceuticals or the like, such as biologics, for the sustained release of medication for greater than six months to overcome difficulties with daily dosing regimes.

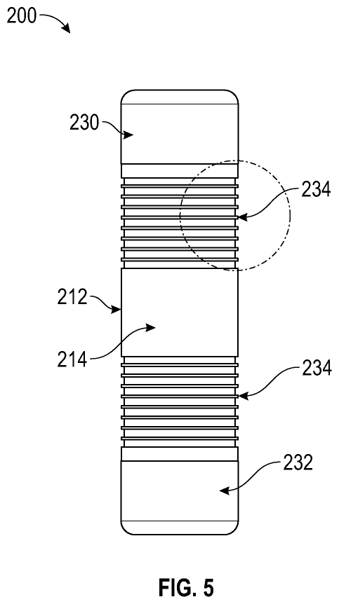

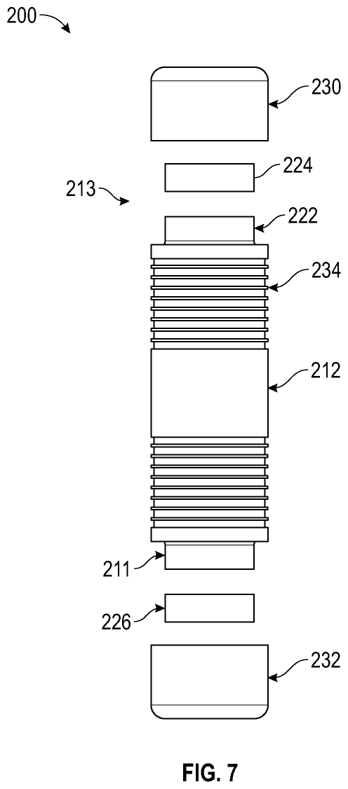

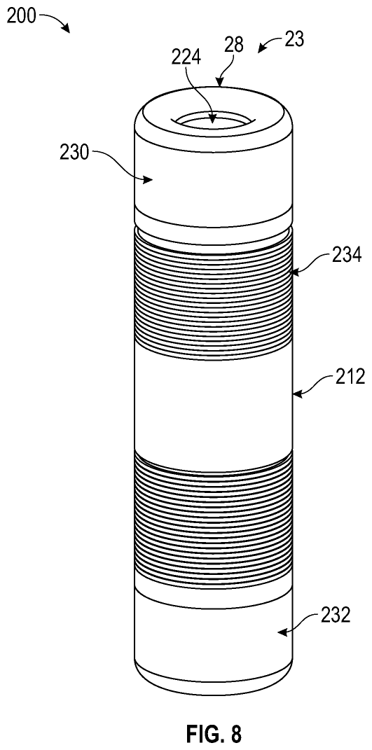

In other embodiments and as shown in FIGS. 5-12, exemplary drug delivery assembly 200 takes the form of a single compartment and single or dual porous membrane based (e.g., porous zinc membrane based) drug delivery assembly 200 for extended drug 28 delivery (e.g., via passive diffusion) and/or tunability or the like.

Example drug delivery assembly 200 includes a housing 212 that extends from a first end 211 to a second end 213. In exemplary embodiments, the housing 212 is substantially tubular or substantially cylindrical with a hollow interior, although the present disclosure is not limited thereto. Rather, it is noted that housing 212 can take a variety of shapes and/or forms.

It is noted that housing 212 can be fabricated from a variety of materials. For example, housing 212 can be fabricated from a metal (e.g., magnesium, zinc, titanium, iron, stainless steel, or an alloy thereof). Housing 212 can also be fabricated from a biodegradable plastic (e.g., PLA or PLLA, etc.).

In non-limiting examples, housing 212 takes the form of a tube or substantially cylindrical shape, as similarly discussed above. In example and non-limiting embodiments, the housing 212 can extend 1.02 cm or 1.29 cm or 1.63 cm from first end 211 to second end 213. In example and non-limiting embodiments, the housing 212 can have a wall thickness of 0.1016 cm.

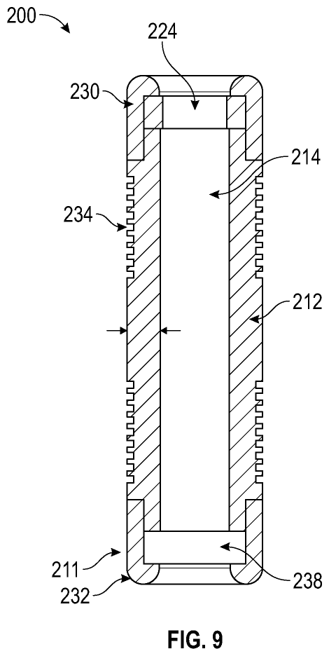

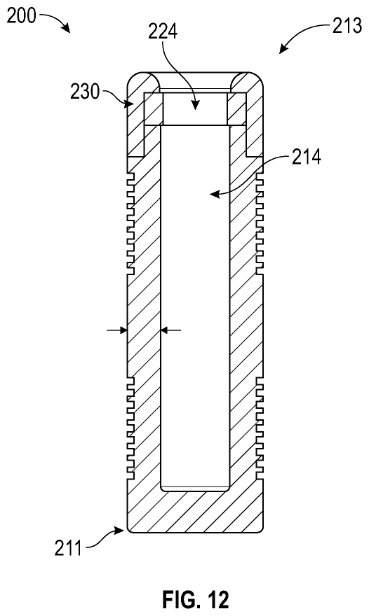

Example housing 212 defines a first compartment 214 (FIGS. 9 and 12). An opening 222 in housing 212 is positioned at the second end 213 of housing 212 of assembly 200. In general, opening 222 can be in communication with an area 23 that is external to the housing 212 of assembly 200 (e.g., an area 23 such as, for example, subcutaneous tissue of a body of a patient, after assembly 200 is positioned in the patient). In example and non-limiting embodiments, the first compartment 214 can have a total internal volume of 0.25 ml or 0.50 ml or 1.00 ml.

In certain embodiments, a first porous membrane 224 is positioned proximal to the opening 222, and a second porous membrane 226 is positioned proximal to first end 211, as discussed further below.

In some embodiments, the first porous membrane 224 can be attached and/or secured relative to the opening 222 of housing 212 via a first end cap 230 attached and/or secured to second end 213, and the second porous membrane 226 (if present) can be attached and/or secured relative to the first end 211 of housing 212 via a second end cap 232 attached and/or secured to first end 211. It is noted that first end cap 230 can include a hood-like feature that is designed to prevent fibrosis from impeding the diffusion properties of the first porous membrane 224. In example and non-limiting embodiments, the first end cap 230 can have an outer diameter of 0.34 cm or 0.43 cm or 0.54 cm.

Interior cavity of first compartment 214 can be filled with a drug solution 28 or the like (e.g., based on a formulation a user), as discussed further below.

An exemplary range of the mean pore size of the first and second porous membranes 224, 226 can each be between 0.10 to 100 μm, although the present disclosure is not limited thereto.

In non-limiting examples, each porous membrane 224, 226 utilized to regulate the mass transfer of the drug 28 can be fabricated from zinc, titanium, polyether ether ketone, or another applicable or suitable biomaterial. Each porous membrane 224, 226 can be in the shape of a flat cylinder or thin needle, with diameters between 0.25 to 10 mm and thicknesses between 0.25 to 10 mm, although the present disclosure is not limited thereto.

In general, compartment 214 is configured and dimensioned to house drug or active agent particles 28.

As such, FIGS. 5-8 depict an exemplary drug delivery assembly 200, with the assembly 200 having compartment 214, and having first and second porous membranes 224, 226 to regulate the delivery of a compound/drug or active agent particles 28 to area 23 that is external to assembly 200.

In exemplary embodiments, it is noted that the assembly 200 is configured and dimensioned to be implanted in a body of a patient or the like (e.g., the subcutaneous layer of a body of a patient or the like for delivery of drug 28). It is also noted that the assembly 200 can be located in other/different areas of the body or the like.

In use, exemplary assembly 200 can be utilized for the extended drug 28 delivery of pharmaceuticals or the like, such as biologics, for the sustained release of medication for greater than six months to overcome difficulties with daily dosing regimes.

As noted, FIGS. 5-8 depict an exemplary drug delivery assembly 200, with the assembly 200 having compartment 214, and having first and second porous membranes 224, 226 secured to housing 212 via first and second end caps 230, 232. The second porous membrane 226 can be attached to a filling apparatus or the like in order to fill compartment 214 with drug or active agent particles 28, either before or after assembly 200 is implanted. The second porous membrane 226 can be closed after filling compartment 214 with drug 28 (e.g., closed with a hydroxyapatite cement or other biocompatible cement).

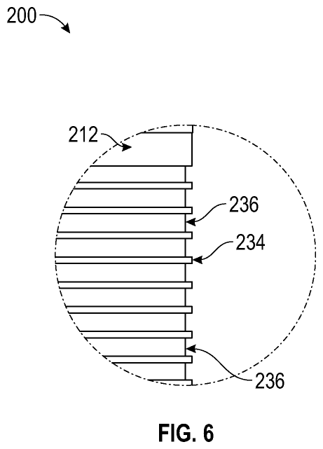

As shown in FIGS. 5-12, the housing 212 includes at least one grooved or threaded section 234 (e.g., two or more sections 234). Each grooved or threaded section 234 includes a plurality of tissue grooves 236 (FIG. 6). As such, the outside surface of the housing 212 is lined with grooved or threaded sections 234 having a plurality of tissue grooves 236, with the tissue grooves 236 promoting the adhesion of tissue to the assembly 200 (e.g., to prevent assembly 200 migration within the body). The grooved or threaded sections 234 modify the surface roughness of the exterior of the assembly 200 to promote tissue growth around the assembly 200 to hold it in place. As such, the grooved or threaded sections 234 along housing 212 act to promote tissue adhesion and reduce implant assembly 200 migration. In one embodiment, the thread feature 234 conforms to a #6-40 thread classified by ASME B1.1.

Features such as hooks/loops can be added to housing 212 to promote suturing of the assembly 200 (e.g., to a piece of the dermal layer). Additionally, polymeric coatings can be applied to housing 212 to modify the chemical and/or physical properties at the surface of the housing 212 of assembly 200. It is noted that a large range of surface roughening processes, such as various types of threading, sanding, and other modification methods can also be utilized on housing 212 to promote the adhesion of tissue to the assembly 200. Preferably, raised surface features in the range of 200 to 500 microns can be created on the surface of the housing 212 to promote adhesion of tissue to the assembly 200.

It is noted that an entirely dissolvable design of housing 212 can be fabricated from zinc, and a refillable design of housing 212 can be fabricated from titanium, although the present disclosure is not limited thereto.

FIG. 9 depicts an exemplary drug delivery assembly 200, with the assembly 200 having compartment 214, and having first porous membrane 224 secured to housing 212 via first end cap 230, and with a septum member 238 secured to first end 211 via second end cap. The septum member 238 can be attached to a filling apparatus or the like in order to fill compartment 214 with drug or active agent particles 28, either before or after assembly 200 is implanted. The septum member 238 can be closed, if desired, after filling compartment 214 with drug (e.g., closed with a hydroxyapatite cement or other biocompatible cement).

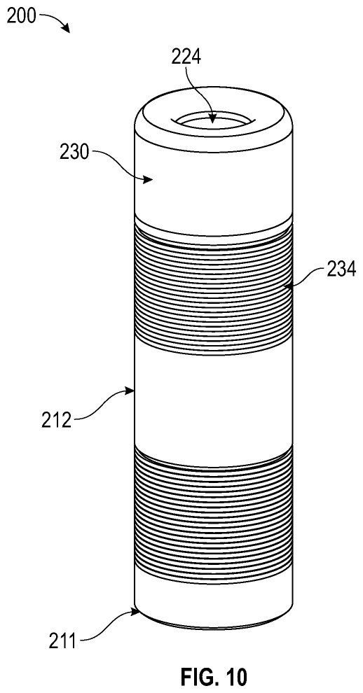

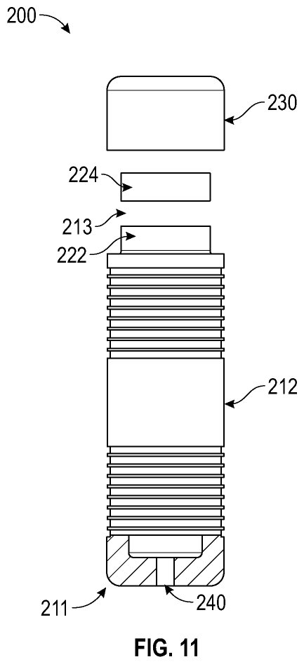

FIGS. 10-11 depict an exemplary drug delivery assembly 200, with the assembly 200 having compartment 214, and having first porous membrane 224 secured to housing 212 via first end cap 230, and with a drug loading port 240 positioned at first end 211. The drug loading port 240 can be attached to a filling apparatus or the like in order to fill compartment 214 with drug or active agent particles 28, either before or after assembly 200 is implanted. The drug loading port 240 can be closed, if desired, after filling compartment 214 with drug 28 (e.g., closed with a hydroxyapatite cement or other biocompatible cement).

FIG. 12 depicts an exemplary drug delivery assembly 200, with the assembly 200 having compartment 214, and having first porous membrane 224 secured to housing 212 via first end cap 230, and with first end 211 being closed off. The compartment 214 can be filled with drug or active agent particles 28 via second end 213 (e.g., before membrane 224 is secured to housing 212).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A drug delivery assembly comprising:
a housing that extends from a first end to a second end, the housing defining a first compartment and a second compartment;
a first opening in the housing in communication with the first and second compartments, the first opening located at a position between the first and second ends of the housing;
a second opening in the housing, the second opening positioned at the second end of the housing, with the second opening in communication with an area that is external to the housing; and
a first porous membrane positioned in the first opening, and a second porous membrane positioned in the second opening,
wherein the first porous membrane has a smaller mean pore size relative to the mean pore size of the second porous membrane.

2. The drug delivery assembly of claim 1, wherein the first and second porous membranes are fabricated out of zinc, magnesium or iron or combinations thereof.

3. The drug delivery assembly of claim 1, wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biomaterials or combinations thereof.

4. The drug delivery assembly of claim 1, wherein the housing is substantially tubular or substantially cylindrical.

5. The drug delivery assembly of claim 1, wherein the housing is fabricated from a metal or from plastic.

6. The drug delivery assembly of claim 1, wherein the housing is fabricated from a material selected from the group consisting of zinc, iron, magnesium, titanium, metal alloys, polylactic acid, poly(lactic-co-glycolic acid) or combinations thereof.

7. The drug delivery assembly of claim 1, wherein the area that is external to the housing includes subcutaneous tissue.

8. The drug delivery assembly of claim 1, wherein a range of a mean pore size of the first porous membrane is between 0.05 to 20 μm.

9. The drug delivery assembly of claim 1, wherein a range of a mean pore size of the second porous membrane is between 1 to 100 μm.

10. The drug delivery assembly of claim 1, wherein the first and second porous membranes are fabricated out of titanium or polyether ether ketone.

11. The drug delivery assembly of claim 1, wherein the first and second porous membranes are in the shape of a flat cylinder or a thin needle.

12. The drug delivery assembly of claim 1, wherein the first and second compartments are configured and dimensioned to house drug or active agent particles.

13. The drug delivery assembly of claim 1, wherein an internal volume of the first compartment is about 0.10 to 2.5 mL, and wherein an internal volume of the second compartment is about 0.10 to 2.5 mL.

14. The drug delivery assembly of claim 1, wherein a total internal volume of the first compartment and of the second compartment added together ranges from about 0.20 mL to about 5 mL.

15. The drug delivery assembly of claim 1, wherein the diameter of the housing is about 0.20 to 15 mm, and the length of the housing is about 0.50 to 15 cm.

16. A drug delivery assembly comprising:
a housing that extends from a first end to a second end, the housing defining a first compartment;
an opening in the housing in communication with an area that is external to the housing;
a first porous membrane positioned proximal to the opening; and
a second porous membrane positioned in the opening.

17. The drug delivery assembly of claim 16, wherein the first and second porous membranes are joined or attached together.

18. The drug delivery assembly of claim 16, wherein the first and second porous membranes are fabricated out of biodegradable metals, such as zinc, magnesium or iron or combinations thereof.

19. The drug delivery assembly of claim 16, wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biocompatible materials, biodegradable materials, or combinations thereof.

20. The drug delivery assembly of claim 16, wherein a range of a mean pore size of the first porous membrane is between 0.05 to 20 μm.

21. The drug delivery assembly of claim 16, wherein a range of a mean pore size of the second porous membrane is between 1 to 100 μm.

22. The drug delivery assembly of claim 16, wherein the first compartment is configured and dimensioned to house drug or active agent particles.

23. A method for utilizing a drug delivery assembly comprising:
providing a housing that extends from a first end to a second end, the housing defining a first compartment and a second compartment;
providing a first opening in the housing in communication with the first and second compartments, the first opening located at a position between the first and second ends of the housing;
providing a second opening in the housing, the second opening positioned at the second end of the housing, with the second opening in communication with an area that is external to the housing; and
positioning a first porous membrane in the first opening, and a positioning a second porous membrane in the second opening; and
providing drug or active agent particles to the first or second compartment,
wherein the first porous membrane has a smaller mean pore size relative to the mean pore size of the second porous membrane.

24. The method of claim 23, wherein the first and second porous membranes are fabricated out of zinc, magnesium or iron.

25. The method of claim 23, wherein the first and second porous membranes are fabricated out of a material selected from the group consisting of zinc, iron, magnesium, titanium, PEEK, metal alloys, polylactic acid, poly(lactic-co-glycolic acid), polyether ether ketone, biomaterials or combinations thereof.

26. A drug delivery assembly comprising:
a housing that extends from a first end to a second end, the housing defining a first compartment;
an opening in the housing in communication with an area that is external to the housing;
a first porous membrane positioned proximal to the opening and secured to the housing via a first end cap; and
a textured section on the housing, the textured section including raised textures on the housing of from 200 to 500 microns.

27. The drug delivery assembly of claim 26, wherein the first end cap includes a hood-like feature designed to prevent fibrosis from impeding the diffusion properties of the first porous membrane.

28. The drug delivery assembly of claim 26 further comprising a second porous membrane secured to the first end of the housing via a second end cap.

29. The drug delivery assembly of claim 26, wherein the first compartment is configured and dimensioned to house drug or active agent particles; and wherein the area that is external to the housing includes subcutaneous tissue; and wherein a range of a mean pore size of the first porous membrane is between 0.10 to 100 μm.

30. The drug delivery assembly of claim 26, wherein the housing is fabricated from zinc or titanium.

31. The drug delivery assembly of claim 26 further comprising a septum member secured to the first end of the housing via a second end cap.

32. The drug delivery assembly of claim 26 further comprising a drug loading port positioned at the first end of the housing.

33. The drug delivery assembly of claim 26, wherein the first end of the housing is closed off.

34. The drug delivery assembly of claim 26, wherein the textured section on the housing comprises a grooved or threaded section, the grooved or threaded section having a plurality of tissue grooves.
